(19) United States
(12) Patent Application Publication
(10) Pub. No.: US 2008/0171662 A1
(43) Pub. Date: Jul. 17, 2008

(54) Aminocarbonyl-Substituted Thiensulfonylamino(Thio)Carbonyl-Triazolin(Ethi)Ones, Processes For Their Preparation And Their Use

(76) Inventors: Ernst R. F. Gesing, Erkrath-Hochdahl (DE); Thomas Geller, Odenthal (DE); Dieter Feucht, Eschborn (DE); Heinz Kehne, Hofheim (DE); Thomas Auler, Leichlingen (DE); Martin Hills, Idstein (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/658,541

(22) PCT Filed: Jul. 13, 2005
(86) PCT No.: PCT/EP2005/007581

§ 371 Date: Jan. 25, 2007

(30) Foreign Application Priority Data
Jul. 28, 2004 (DE) 10 2004 036 552.0

(51) Int. Cl. A01N 43/653; C07D 249/12; A01P 13/00

(52) U.S. Cl. 504/273; 548/263.4

(57) ABSTRACT

Aminocarbonyl-substituted thiensulfonylamino(thio)carbonyl-triazolin(ethi)ones, processes for their preparation and their use

The invention relates to novel substituted thien-3-ylsulfonylamino(thio)carbonyl-triazolin(ethi)ones of the formula (I),

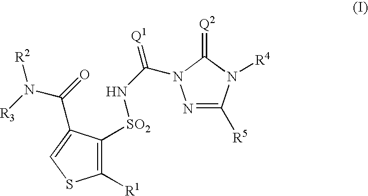

in which
Q¹, Q², R¹, R², R³, R⁴ and R⁵ are as defined in the description, and also to salts of compounds of the formula (I), to processes for their preparation and to their use as herbicides.

Aminocarbonyl-substituted thiensulphonylamino (thio) carbonyltriazolin (ethi) ones, methods for production and use thereof

The invention related to novel aminocarbonyl-substituted thien-3-ylsulfonylamino(thio)-carbonyl-triazolin(ethi)ones, to processes for their preparation and to their use as herbicides.

It is already known that certain substituted thienylsulfonylamino(thio)carbonyl-triazolin-(ethi)ones have herbicidal properties (cf. WO-A-97/16449, WO-A-01/05788). However, the activity of these known compounds is not entirely satisfactory.

This invention now provides the novel substituted thien-3-ylsulfonylamino(thio)carbonyl-triazolin(ethi)ones of the formula (I)

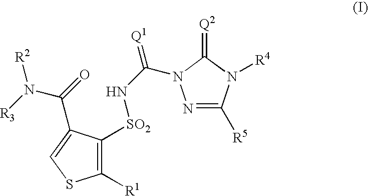

in which
Q¹ is O (oxygen) or S (sulfur),
Q² is O (oxygen) or S (sulfur),
R¹ is hydrogen, cyano, nitro, halogen, is in each case optionally cyano-, halogen- or C₁-C₄-alkoxy-substituted alkyl, alkoxy, alkoxycarbonyl, alkylthio, alkylsulfinyl or alkylsulfonyl having in each case 1 to 6 carbon atoms in the alkyl group, or is in each case optionally cyano- or halogen-substituted alkenyl, alkynyl, alkenyloxy or alkynyloxy having in each case 2 to 6 carbon atoms in the alkenyl or alkynyl group,
R² is hydrogen, is in each case optionally cyano-, halogen-, C₁-C₄-alkylthio-, C₁-C₄-alkylsulfinyl-, C₁-C₄-alkylsulfonyl- or C₁-C₄-alkoxy-substituted alkyl, alkoxy-carbonyl, alkylsulfonyl having in each case 1 to 10 carbon atoms in the alkyl group, or is in each case optionally cyano-, halogen- or C₃-C₆-cycloalkyl-substituted cycloalkyl or cycloalkylalkyl (for example monocycloalkylalkyl or dicycloalkylalkyl) having 3 to 6 carbon atoms in the cycloalkyl ring and 1 to 3 carbon atoms in the alkyl group,
R³ is hydrogen or is alkyl having 1 to 6 carbon atoms, or
R² and R³ together are optionally branched alkanediyl having 3 to 7 carbon atoms, where in the alkanediyl chain optionally one methylene group is replaced by an oxygen or nitrogen atom,
R⁴ is hydrogen, hydroxyl, amino, cyano, is C₂-C₁₀-alkylideneamino, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, bromine-, cyano-, C₁-C₄-alkoxy- or C₁-C₄-alkoxy-carbonyl-substituted alkoxy, alkylamino or alkylcarbonylamino having in each case 1 to 6 carbon atoms in the alkyl group, is alkenyloxy (for example monocycloalkylalkyl or dicycloalkyl alkyl) having 3 to 6 carbon atoms, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkylamino or cycloalkylalkyl (for example monocycloalkylalkyl or dicycloalkylalkyl) having in each case 3 to 6 carbon atoms in the alkyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl- and/or C₁-C₄-alkoxy-substituted aryl or arylalkyl having in each case 6 or 10 carbon atoms in the aryl group and, if appropriate, 1 to 4 carbon atoms in the alkyl moiety, and
R⁵ is hydrogen, hydroxyl, mercapto, amino, cyano, fluorine, chlorine, bromine, iodine, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, cyano-, C₁-C₄-alkoxy- or C₁-C₄-alkoxy-carbonyl-substituted alkoxy, alkylthio, alkylamino or alkylcarbonylamino having in each case 1 to 6 carbon atoms in the alkyl group, is alkenyloxy, alkynyloxy, alkenylthio, alkynylthio, alkenylamino or alkynylamino having in each case 3 to 6 carbon atoms in the alkenyl or alkynyl group, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkenyl, cycloalkyloxy, cycloalkylthio, cycloalkylamino, cycloalkylalkyl, (for example monocycloalkylalkyl or dicycloalkylalkyl) cycloalkylalkoxy, cycloalkylalkylthio or cycloalkylalkylamino having in each case 3 to 6 carbon atoms in the cycloalkyl or cycloalkenyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl-, C₁-C₄-alkoxy- and/or C₁-C₄-alkoxy-carbonyl-substituted aryl, arylalkyl, aryloxy, arylalkoxy, arylthio, arylalkylthio, arylamino or arylalkylamino having in each case 6 or 10 carbon atoms in the aryl group and, if appropriate, 1 to 4 carbon atoms in the alkyl moiety
and salts of the compounds of the formula (I).

Saturated or unsaturated hydrocarbon groupings, such as alkyl, alkanediyl, alkenyl or alkynyl, are in each case straight-chain or branched as far as this is possible—including in combination with heteroatoms, such as in alkoxy.

Optionally substituted radicals may be mono- or polysubstituted, where in the case of polysubstitution the substituents can be identical or different.

Preferred substituents or ranges of the radicals present in the formulae given above and below are defined below.

Q¹ is preferably O (oxygen).
Q² is preferably O (oxygen).
R¹ is preferably hydrogen, cyano, fluorine, chlorine, bromine, is preferably in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy, methoxycarbonyl, ethoxycarbonyl, n- or i-propoxycarbonyl, methylthio, ethylthio, n- or i-propylthio, methylsulfinyl, ethylsulfinyl, methylsulfonyl or ethylsulfonyl, or is preferably in each case optionally cyano-, fluorine- or chlorine-substituted propenyl, butenyl, propynyl, butynyl, propenyloxy, butenyloxy, propynyloxy or butynyloxy.
R¹ is particularly preferably fluorine, chlorine, bromine, is particularly preferably in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy.
R¹ is very particularly preferably methyl, ethyl, n- or i-propyl.
R² is preferably hydrogen, is preferably in each case optionally cyano-, halogen-, methoxy- or ethoxy-substituted methyl, ethyl, n- or i-propyl, n-, i-, s- or t-butyl, methoxycarbonyl, ethoxycarbonyl, n-propoxycarbonyl, i-propoxycarbonyl, methyl-sulfonyl or ethylsulfonyl, or is preferably in each case optionally cyano- or halogen-substituted cyclopropyl, cyclobutyl, cyclopentyl, cyclohexyl, cyclopropylmethyl, cyclobutylmethyl, cyclopentylmethyl, cyclohexylmethyl, dicyclopropylmethyl, dicyclobutylmethyl, dicyclopentylmethyl or dicyclohexylmethyl or -C(C₁-C₄-alkyl)₂-CH₂-S(O)n-(C₁-C₄-alkyl) where n=0, 1 or 2.
R² is very particularly preferably hydrogen, methyl, ethyl, n- or i-propyl, cyclopropyl, cyclobutyl, cyclopentyl, cyclohexyl, cyclopropylmethyl or dicyclopropylmethyl.
R² is most preferably hydrogen, methyl, ethyl, n- or i-propyl or cyclopropyl.
R³ is preferably hydrogen, methyl, ethyl, n- or i-propyl.
R³ is very particularly preferably hydrogen or methyl.
R² and R³ together are also preferably trimethylene (propane-1 ,3-diyl), tetramethylene (butane-1 ,4-diyl), pentamethylene (pentane-1,5-diyl) or -CH₂-CH₂-O-CH₂-CH₂-.
R² and R³ together are also particularly preferably tetramethylene (butane-1,4-diyl) or pentamethylene (pentane-1,5-diyl).
R⁴ is preferably hydrogen, hydroxyl, amino, is preferably in each case optionally fluorine-, chlorine-, cyano-, methoxy- or ethoxy-substituted methyl, ethyl, n- or i-propyl, n-, i-, s- or t-butyl, is preferably in each case optionally fluorine-, chlorine- and/or bromine-substituted ethenyl, propenyl, butenyl, propynyl or butynyl, is preferably in each case optionally fluorine-, chlorine-, cyano-, methoxy- or ethoxy-substituted methoxy, ethoxy, n- or i-propoxy, n-, i-, s- or t-butoxy, methylami no, ethylamino, n- or i-propylamino, n-, i-, s- or t-butylamino, is preferably propenyloxy or butenyloxy, is preferably dimethylamino or diethylamino, is preferably in each case optionally fluorine-, chlorine-, methyl-and/or ethyl-substituted cyclopropyl, cyclobutyl, cyclopentyl, cyclohexyl, cyclopropylamino, cyclobutylamino, cyclopentylamino, cyclohexylamino, cyclopropylmethyl, cyclobutylmethyl, cyclopentylmethyl, cyclohexylmethyl, dicyclopropylmethyl, dicyclobutylmethyl, dicyclopentylmethyl or dicyclohexylmethyl, or is preferably in each case optionally fluorine-, chlorine-, methyl-, trifluoromethyl- and/or methoxy-substituted phenyl or benzyl.
R⁴ is particularly preferably methyl, ethyl, n- or i-propyl or cyclopropyl.
R⁵ is preferably hydrogen, hydroxyl, mercapto, amino, cyano, fluorine, chlorine, bromine, is preferably in each case optionally fluorine-, chlorine-, cyano-, methoxy-, ethoxy-, n- or i-propoxy-, acetyl-, propionyl-, n- or i-butyroyl-, methoxycarbonyl-, ethoxycarbonyl-, n- or i-propoxycarbonyl-substituted methyl, ethyl, n- or i-propyl, n-, i-, s- or t-butyl, is preferably in each case optionally fluorine-, chlorine- and/or bromine-substituted ethenyl, propenyl, butenyl, ethynyl, propynyl or butynyl, is preferably in each case optionally fluorine-, chlorine-, cyano-, methoxy-, ethoxy-, n- or i-propoxy-, methoxycarbonyl-, ethoxycarbonyl-, n- or i-propoxycarbonyl-substituted methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, acetylamino or propionylamino, is preferably propenyloxy, butenyloxy, ethynyloxy, propynyloxy, butynyloxy, propenylthio, butenylthio, propynylthio, butynylthio, propenylamino, butenylamino, propynylamino or butynylamino, is preferably dimethylamino, diethylamino or dipropylamino, is preferably in each case optionally fluorine-, chlorine-, methyl- and/or ethyl-substituted cyclopropyl, cyclobutyl, cyclopentyl, cyclohexyl, cyclopentenyl, cyclohexenyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethylthio, cyclo-butylmethylthio, cyclopentylmethylthio, cyclohexylmethylthio, cyclo-propylmethylamino, cyclobutylmethylamino, cyclopentylmethylamino or cyclohexylmethylamino, or is preferably in each case optionally fluorine-, chlorine-, bromine-, methyl-, trifluoromethyl-, methoxy- or methoxycarbonyl-substituted phenyl, benzyl, phenoxy, benzyloxy, phenylthio, benzylthio, phenylamino or benzylamino.
R⁵ is particularly preferably methoxy, ethoxy, n- or i-propoxy, n-, i-, s- or t-butoxy.

The invention preferably also provides the sodium, potassium, magnesium, calcium, ammonium, C₁-C₄-alkylammonium, di-(C₁-C₄-alkyl)ammonium, tri-(C₁-C₄-alkyl)-ammonium, tetra-(C₁-C₄-alkyl)ammonium, tri-(C₁-C₄-alkyl)sulfonium, C₅- or C₆-cycloalkylammonium and di-(C₁-C₂-alkyl)benzylammonium salts of compounds of the formula (I), in which Q¹, Q², R¹, R^{2, R}³, R⁴ and R⁵ have the meaning given above as being preferred.

The general or preferred radical definitions given above apply both to the end products of the formula (I) and, correspondingly, to the starting materials and intermediates required in each case for the preparation. These radical definitions, can be combined with one another as desired, i.e. including combinations between the given ranges of preferred compounds.

Preference according to the invention is given to those compounds of the formula (I) which contain a combination of the meanings given above as being preferred. Particular preference according to the invention is given to those compounds of the formula (I) which contain a combination of the meanings given above as being particularly preferred.

Most preference according to the invention is given to those compounds of the formula (I) which contain a combination of the meanings given above as being very particularly preferred.

The novel substituted thien-3-ylsulfonylamino(thio)carbonyl-triazolin(ethi)ones of the formula (I) have interesting biological properties. In particular, they have strong herbicidal activity.

The novel substituted thien-3-ylsulfonylamino(thio)carbonyl-triazolin(ethi)ones of the formula (I) are obtained when

substituted thiophene-3-sulfonamides of the formula (II)

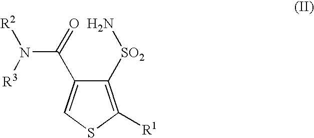

in which
R¹, R² and R³ are as defined above,

are reacted with triazolin(ethi)ones of the formula (III)

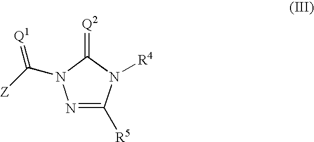

in which
Q¹, Q², R⁴ and R⁵ are as defined above and
Z is halogen, alkoxy, aryloxy or arylalkoxy,
if appropriate in the presence of a reaction auxiliary and if appropriate in the presence of a diluent,
and, if appropriate, converting the compounds of the formula (I) obtained by the process by customary methods into salts.

Using, for example, 2-methyl-4-dimethylaminocarbonyl thiophene-3-sulfonamide and 4,5-dimethoxy-2-phenoxycarbonyl-2,4-dihydro-3H-1,2,4-triazol-3-one as starting materials, the course of the reaction in the process according to the invention can be illustrated by the formula scheme below:

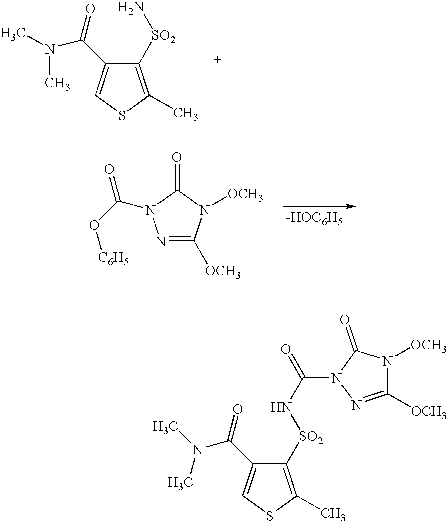

The formula (II) provides a general definition of the substituted thiophene-3-sulfon-amides to be used in the process according to the invention for preparing compounds of the formula (I). In the formula (II), R¹, R² and R³ preferably or in particular have those meanings which have already been given above in connection with the description of the compounds of the formula (I) according to the invention as being preferred or as being particularly preferred for R¹, R² and R³.

Except for the compound 4-sulfamoylthiophene-3-carboxamide (known from U.S. Pat. No. 4,028,373), the substituted thiophene-3-sulfonamides of the formula (II) have hitherto not been disclosed in the literature. From among the compounds of the formula (II), preference is given to those in which R¹ is not hydrogen.

The substituted thiophene-3-sulfonamides of the formula (II) are obtained when substituted thiophene-3-sulfonamides of the formula (IV)

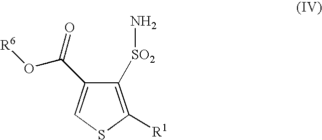

in which
R¹is as defined above und R⁶ is C₁-C₄-alkyl,
are reacted with amines of the formula (V)

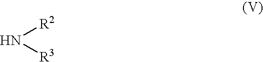

in which R² und R³ are as defined above,
if appropriate in the presence of a diluent and if appropriate at elevated temperatures between 0° C. and 200° C. (cf. the preparation example).

The thiophene-3-sulfonamides of the formula (IV) are known. They can be prepared according to the methods given in WO-A-01/05788 or according to the methods known from the literature.

The formula (III) provides a general definition of the substituted triazolin(ethi)ones further to be used in the process according to the invention for preparing compounds of the formula (I). In the formula (III), Q¹, Q², R⁴ and R⁵ preferably or in particular have those meanings which have already been given above in connection with the description of the compounds of the formula (I) according to the invention as being preferred or as being particularly preferred for Q¹, Q², R⁴ and R⁵.

The starting materials of the formula (III) are known and/or can be prepared by processes known per se (cf. WO 01/05788). Z in formula (III) is preferably chlorine, bromine, methoxy, ethoxy, phenoxy or benzyloxy.

As chemicals for synthesis, the amines of the formula (V) are commercially available or can be prepared by processes known per se.

The process according to the invention for preparing the novel compounds of the formula (I) is preferably carried out using diluents. Suitable diluents are virtually all inert organic solvents. These preferably include aliphatic and aromatic, optionally halogenated hydrocarbons, such as pentane, hexane, heptane, cyclohexane, petroleum ether, petrol, ligroine, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether und diglycol dimethyl ether, tetrahydrofuran and dioxane, ketones, such as acetone, methyl ethyl ketone, methyl isopropyl ketone and methyl isobutyl ketone, esters, such as methyl acetate and ethyl acetate, nitriles, such as, for example, acetonitrile and propionitrile, amides, such as, for example, dimethylformamide, dimethylacetamide and N-methylpyrrolidone, and also dimethyl sulfoxide, tetramethylene sulfone and hexamethylphosphoric triamide.

Suitable as reaction auxiliaries for use in the process according to the invention are all acid binders usually used for such reactions. Preferred are alkali metal hydroxides, such as, for example, sodium hydroxide or potassium hydroxide, alkaline earth metal hydroxides, such as, for example, calcium hydroxide, alkali metal carbonates and alcoxides, such as sodium carbonate and potassium carbonate, sodium and potassium tert-butoxide, furthermore basic nitrogen compounds, such as trimethylamine, triethyl-amine, tripropylamine, tributylamine, diisobutylamine, dicyclohexylamine, ethyidiiso-propylamine, ethyldicyclohexylamine, N,N-dimethylbenzylamine, N,N-dimethylaniline, pyridine, 2-methyl-, 3-methyl-, 4-methyl-, 2,4-dimethyl-, 2,6-dimethyl-, 2-ethyl-, 4-ethyl- and 5-ethyl-2-methylpyridine, 1,5-diazabicyclo[4.3.0]non-5-ene (DBN), 1,8-diazabicyclo-[5.4.0]undec-7-ene (DBU) and 1,4-diazabicyclo[2.2.2]octane (DABCO).

In the process according to the invention, the reaction temperatures can be varied within a relatively large range. In general, the process is carried out at temperatures between −20° C. and +150° C., preferably at temperatures between 0° C. and +100° C.

The process according to the invention is generally carried out under atmospheric pressure. However, it is also possible to carry out the process according to the invention under elevated or reduced pressure.

For carrying out the process according to the invention, the respective starting materials needed are generally employed in approximately equimolar amounts. However, it is also possible to use a relatively large excess of one of the respective components used. The reactions are generally carried out in a suitable diluent in the presence of an acid acceptor, and the reaction mixture is stirred at the respective required temperature for a number of hours. Work-up in the processes according to the invention is in each case carried out by customary methods (cf. the preparation example).

If appropriate, salts may be prepared from the compounds of the formula (I) according to the invention. Such salts are obtained in a simple manner by customary methods for forming salts, for example by dissolving or dispersing a compound of the formula (I) in a suitable solvent, such as, for example, methylene chloride, acetone, tert-butyl methyl ether or toluene, and adding a suitable base. The salts can then be isolated—if appropriate after prolonged stirring—by concentration or filtration with suction.

The active compounds according to the invention can be used as defoliants, desiccants, haulm killers and, especially, as weedkillers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.
Dicotyledonous cross of the genera: Arachis, Beta, Brassica, Cucumis, Cucurbita, Helianthus, Daucus, Glycine, Gossypium, lpomoea, Lactuca, Linum, Lycopersicon, Nicotiana, Phaseolus, Pisum, Solanum, Vicia.
Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, imperata, lschaemum, Leptochloa, Lolium, Monochoria, Panicum, Paspalum, Phalaris, Phleum, Poa, Rottboellia, Sagittaria, Scirpus, Setaria, Sorghum.
Monocotyledonous crops of the genera: Allium, Ananas, Asparagus, Avena, Hordeum, Oryza, Panicum, Saccharum, Secale, Sorghum, Triticale, Triticum, Zea. However, the use of the active compounds according to the invention is in no way restricted to these genera, but also extends in the same manner to other plants.

The active compounds according to the invention are suitable, depending on the concentration, for the total control of weeds, for example on industrial terrain and rail tracks, and on paths and areas with or without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad activity spectrum when used on the soil and on above-ground parts of plants. To a certain extent they are also suitable for the selective control of monocotyledonous and dicotyledonous weeds in monocotyledonous and dicotyledonous crops, both by the pre-emergence and by the post-emergence method. At certain concentrations or application rates, the active compounds according to the invention can also be employed for controlling animal pests and fungal or bacterial plant diseases. If appropriate, they can also be used as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspoemulsion concentrates, natural and synthetic materials impregnated with active compound, and microencapsulations in polymeric materials.

These formulations are produced in a known manner, for example by mixing the active compounds with extenders, that is liquid solvents and/or solid carriers, optionally with the use of surfactants, that is emulsifiers and/or dispersants, and/or foam formers.

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are: for example ammonium salts and ground natural minerals such as kaolins, clays, talc, chalk, quartz, aftapulgite, montmorillonite or diatomaceous earth, and ground synthetic minerals such as finely divided silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as. sawdust, coconut shells, corn cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

For controlling weeds, the active compounds according to the invention, as such or in their formulations, can also be used as mixtures with known herbicides and/or substances which improve the compatibility with crop plants (“safeners”), finished formulations or tank mixes being possible. Also possible are thus mixtures with weedkillers comprising one or more known herbicides and a safener.

Possible components for the mixtures are known herbicides, for example acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (-P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (-P-ethyl), fentrazamide, flamprop (-isopropyl, -isopropyl-L, -methyl), flazasulfuron, florasulam, fluazifop (-P-butyl), fluazolate, flucarbazone (-sodium), flufenacet, flufenpyr, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, -P-methyl), hexazinone, imazamethabenz (-ethyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, ketospiradox, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, penoxysulam, pentoxazone, pethoxamid, phenmedipham, picolinafen, pinoxaden, piperophos, pretilachlor, primisulfuron (-methyl), profluazol, profoxydim, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchiorac, quinmerac, quinoclamine, quizalofop (-P-ethyl, -P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Furthermore suitable for the mixtures are known safeners, for example AD-67, BAS-145138, benoxacor, cloquintocet (-mexyl), cyometrinil, 2,4-D, DKA-24, dichlormid, dymron, fenclorim, fenchlorazol (-ethyl), flurazole, fluxofenim, furilazole, isoxadifen (-ethyl), MCPA, mecoprop (-P), mefenpyr (-diethyl), MG-191, oxabetrinil, PPG-1292, R-29148.

A mixture with other known active compounds, such as fungicides, insecticides, acaricides, nematicides, bird repellents, plant nutrients and soil improvers is also possible.

The active compounds can be used as such, in the form of their formulations or in the use forms prepared therefrom by further dilution, such as ready-to-use solutions, suspensions, emulsions, powders, pastes and granules. The application is carried out in a customary manner, for example by watering, spraying, atomizing, broadcasting.

The active compounds according to the invention can be applied both before and after emergence of the plants. They can also be incorporated into the soil prior to sowing.

The application rate of active compound can vary within a relatively large range. It depends essentially on the nature of the desired effect. In general, the application rates are between 1 g and 10 kg of active compound per hectare of soil, preferably between 5 g and 5 kg per ha.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with specific properties (“traits”) which have been obtained by conventional cultivation, by mutagenesis or else by recombinant DNA techniques. These may be cultivars, biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or widenings of the activity spectrum and/or an increase in the activity of the substances and compositions that can be used according to the invention also in combination with other agrochemical active compounds, better crop plant growth, increased tolerance of the crop plants to high or low temperatures, increased tolerance of the crop plants to drought or to water or soil salt content, increased flowering performance, easier harvesting, accelerated maturation, higher harvest yields, better quality and/or a higher nutritional value of the harvested products, better storage ability and/or processability of the harvested products which exceed the effects which were actually to be expected are possible.

The preferred transgenic plants or plant cultivars (i.e. those obtained by genetic engineering) which are to be treated according to the invention include all plants which, in the genetic modification, received genetic material which imparts particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soya beans, potatoes, cotton, oilseed rape and also fruit plants (with the fruits apples, pears, citrus fruits and grapes), and particular emphasis is given to corn, soya beans, potatoes, cotton and oilseed rape. Traits that are emphasized are in particular increased defense of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIB2, Cry9c, Cry2Ab, Cry3Bb and CryIF and also combinations thereof) (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulfonylureas, glyphosate or phosphinothricin (for example the “PAT” gene). The genes which impart the desired traits in question can also be present in combination with one another in the transgenic plants. Examples of “Bt plants” which may be mentioned are corn varieties, cotton varieties, soybean varieties and potato varieties which are sold under the trade names YIELD GARD® (for example corn, cotton, soybean), KnockOut®) (for example corn), StarLink® (for example corn), Bollgard®) (cotton), Nucotn®) (cotton) and NewLeaf®) (potato). Examples of herbicide-tolerant plants which may be mentioned are corn varieties, cotton varieties and soybean varieties which are sold under the trade names Roundup Ready® (tolerance to glyphosate, for example corn, cotton, soybean), Liberty Link® (tolerance to phosphinothricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned include the varieties sold under the name Clearfield® (for example corn). Of course, these statements also apply to plant cultivars having these genetic traits or genetic traits still to be developed, which cultivars will be developed and/or developed and/or marketed in the future.

The plants listed can be treated according to the invention in a particularly advantageous manner with the compounds of the formula I or the active compound mixtures according to the invention, where, in addition to the effective control of the weed plants, the abovementioned synergistic effects with the transgenic plants or plant cultivars occur. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

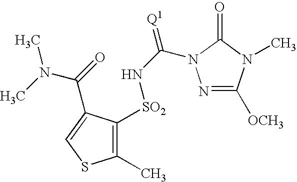

0.67 g (2.7 mmol) of 5-methoxy4-methyl-2-phenoxycarbonyl-2,4-dihydro-3H-1,2,4-triazol-3-one are dissolved in 40 ml of acetonitrile, and 0.8 g (3.2 mmol) of 4-dimethylaminocarbonyl-2-methylthiophene-3-sulfonamide and 0.49 g (3.2 mmol) of 1,8-diazabicyclo-[5,4,0]-undec-7-ene (DBU) are added successively at room-temperature (about 20° C.). The reaction mixture is stirred at room temperature overnight and then concentrated under reduced pressure. The residue is taken up in methylene chloride, washed successively with 5% strength hydrochloric acid and with water, dried with sodium sulfate and filtered. The filtrate is concentrated under water pump vacuum and the crystalline product is isolated by filtration with suction.

This gives 0.65 g (60% of theory) of 5-ethoxy-4-methyl-2-[(4-dimethylaminocarbonyl-2-methylthien-3-yl)sulfonylaminocarbonyl]-2,4-dihydro-3H-1,2,4-triazol-3-one of melting point 162° C.

Analogously to example 1 and in accordance with the general description of the preparation process according to the invention, it is also possible to prepare, for example, the compounds of the formula (I) listed in table 1 below.

Abbreviations to tables 1 and 2:

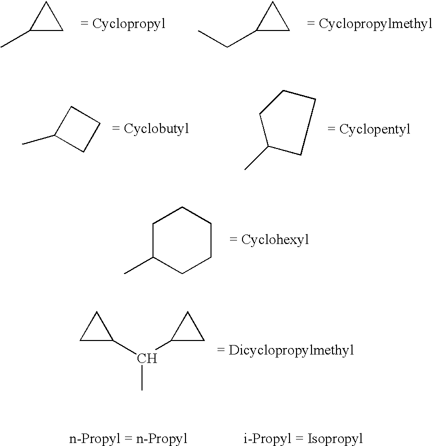

TABLE 1
Examples of compounds of the formula (I)

| | |
|---|---|
| | (I) |
| 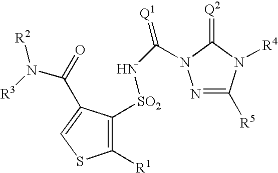 | |

| Ex. | | | | | | | | Melting point |
|---|---|---|---|---|---|---|---|---|
| No. | Q¹ | Q² | R¹ | R² | R³ | R⁴ | R⁵ | (° C.) |
| 2 | O | O | CH₃ | H | H | CH₃ | OCH₃ | 188 |
| 3 | O | O | CH₃ | H | H | CH₃ | OCH₂CH₃ | 172 |
| 4 | O | O | CH₃ | H | H | CH₃ | O-(n-propyl) | 184 |
| 5 | O | O | CH₃ | H | H | CH₃ | O-(i-propyl) | 151 |
| 6 | O | O | CH₃ | H | H | 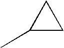 | OCH₃ | 163 |
| 7 | O | O | CH₃ | H | H | 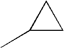 | OCH₂CH₃ | 190 |
| 8 | O | O | CH₃ | H | H | 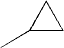 | O-(n-propyl) | 194 |
| 9 | O | O | CH₃ | H | H | 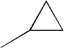 | O-(i-propyl) | 165 |
| 10 | O | O | CH₃ | CH₃ | CH₃ | 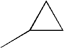 | OCH₂CH₃ | 118 |
| 11 | O | O | CH₃ | CH₃ | H | CH₃ | OCH₃ | 177 |
| 12 | O | O | CH₃ | CH₃ | H | CH₃ | OCH₂CH₃ | 84 |
| 13 | O | O | CH₃ | CH₃ | H | 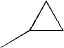 | OCH₂CH₃ | 77 |
| 14 | O | O | CH₃ | CH₃ | CH₃ | CH₃ | OCH₂CH₃ | 120 |
| 15 | O | O | CH₃ | CH₂CH₃ | H | CH₃ | OCH₃ | 151 |
| 16 | O | O | CH₃ | CH₂CH₃ | H | CH₃ | OCH₂CH₃ | 180 |
| 17 | O | O | CH₃ | n-propyl | H | CH₃ | OCH₃ | 124 |
| 18 | O | O | CH₃ | n-propyl | H | CH₃ | OCH₂CH₃ | 193 |
| 19 | O | O | CH₃ | n-propyl | H | 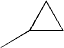 | OCH₂CH₃ | 64 |
| 20 | O | O | CH₃ | i-propyl | H | CH₃ | OCH₃ | 98 |
| 21 | O | O | CH₃ | i-propyl | H | CH₃ | OCH₂CH₃ | 190 |
| 22 | O | O | CH₃ | i-propyl | H | 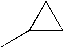 | OCH₂CH₃ | 162 |
| 23 | O | O | CH₃ | 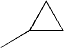 | H | CH₃ | OCH₃ | 188 |
| 24 | O | O | CH₃ | 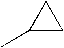 | H | CH₃ | OCH₂CH₃ | 160 |
| 25 | O | O | CH₃ | 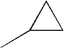 | H | 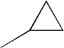 | OCH₂CH₃ | 168 |
| 26 | O | O | CH₃ | CH₂CH₃ | H | 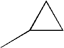 | OCH₂CH₃ | 78 |
| 27 | O | O | CH₃ | —CH₂CH₂CH₂CH₂— | | CH₃ | OCH₃ | 195 |
| 28 | O | O | CH₃ | —CH₂CH₂CH₂CH₂— | | CH₃ | OCH₂CH₃ | 153 |
| 29 | O | O | CH₃ | —CH₂CH₂CH₂CH₂— | | CH₃ | O-(i-propyl) | 128 |
| 30 | O | O | CH₃ | —CH₂CH₂CH₂CH₂— | | 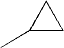 | OCH₃ | 174 |
| 31 | O | O | CH₃ | —CH₂CH₂CH₂CH₂— | | 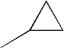 | OCH₂CH₃ | 110 |
| 32 | O | O | CH₃ | —CH₂CH₂CH₂CH₂— | | 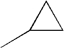 | O-(n-propyl) | 160 |
| 33 | O | O | CH₃ | —CH₂CH₂CH₂CH₂CH₂— | | CH₃ | OCH₃ | 190 |
| 34 | O | O | CH₃ | —CH₂CH₂CH₂CH₂CH₂— | | 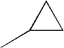 | OCH₂CH₃ | 127 |
| 35 | O | O | CH₃ | 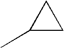 | H | CH₃ | O-(n-propyl) | 172 |
| 36 | O | O | CH₃ | 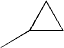 | H | CH₃ | O-(i-propyl) | 175 |
| 37 | O | O | CH₃ | 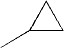 | H | 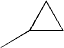 | OCH₃ | 119 |
| 38 | O | O | CH₃ | 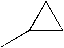 | H | 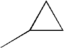 | O-(n-propyl) | 120 |
| 39 | O | O | CH₃ | 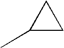 | H | 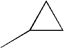 | O-(i-propyl) | 139 |
| 40 | O | O | CH₃ | 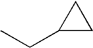 | H | CH₃ | OCH₃ | 130 |
| 41 | O | O | CH₃ | 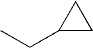 | H | CH₃ | OCH₂CH₃ | 172 |
| 42 | O | O | CH₃ | 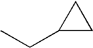 | H | CH₃ | O-(n-propyl) | 136 |
| 43 | O | O | CH₃ | 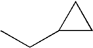 | H | CH₃ | O-(i-propyl) | 197 |
| 44 | O | O | CH₃ | 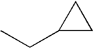 | H | 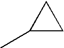 | OCH₃ | 168 |
| 45 | O | O | CH₃ | 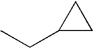 | H | 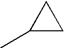 | OCH₂CH₃ | 144 |
| 46 | O | O | CH₃ | 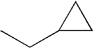 | H | 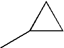 | O-(n-propyl) | 143 |
| 47 | O | O | CH₃ | 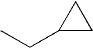 | H | 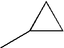 | O-(i-propyl) | 195 |
| 48 | O | O | CH₃ | 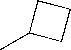 | H | CH₃ | OCH₃ | 150 |
| 49 | O | O | CH₃ | 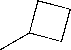 | H | CH₃ | OCH₂CH₃ | 166 |
| 50 | O | O | CH₃ | 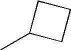 | H | CH₃ | O-(n-propyl) | 155 |
| 51 | O | O | CH₃ | 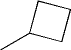 | H | CH₃ | O-(i-propyl) | 166 |
| 52 | O | O | CH₃ | 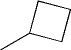 | H | 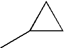 | OCH₃ | 192 |
| 53 | O | O | CH₃ | 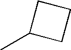 | H | 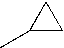 | OCH₂CH₃ | 183 |
| 54 | O | O | CH₃ | 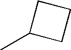 | H | 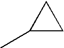 | O-(n-propyl) | 169 |
| 55 | O | O | CH₃ | 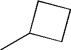 | H | 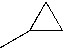 | O-(i-propyl) | 187 |
| 56 | O | O | CH₃ | 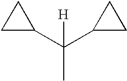 | H | CH₃ | OCH₃ | 148 |
| 57 | O | O | CH₃ | 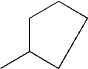 | H | CH₃ | OCH₃ | 174 |
| 78 | O | O | CH₃ | 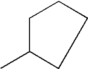 | H | CH₃ | OCH₂CH₃ | 170 |
| 79 | O | O | CH₃ | 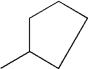 | H | CH₃ | O-(i-propyl) | 169 |
| 80 | O | O | CH₃ | 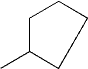 | H | 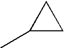 | OCH₃ | 185 |
| 81 | O | O | CH₃ | 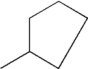 | H | 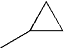 | OCH₂CH₃ | 174 |
| 82 | O | O | CH₃ | 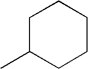 | H | CH₃ | OCH₃ | 176 |
| 83 | O | O | CH₃ | 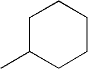 | H | CH₃ | OCH₂CH₃ | 169 |
| 84 | O | O | CH₃ | 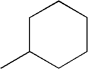 | H | CH₃ | O-(i-propyl) | 182 |
| 85 | O | O | CH₃ | 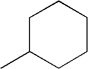 | H | 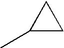 | OCH₃ | 195 |
| 86 | O | O | CH₃ | 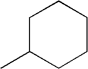 | H | 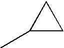 | OCH₂CH₃ | 182 |

Starting materials of the formula (II):

### Example (II-1)

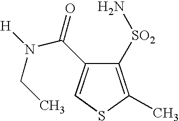

In an autoclave, 38.3 g (850 mmol) of ethylamine are added to a suspension of 20.0 g (85 mmol) of 4-methoxycarbonyl-2-methylthiophene-3-sulfonamide in 120 ml of toluene. The mixture is heated at 100° C. for 20 hours. After cooling, the solvent is distilled off under water pump vacuum, the residue is digested with petroleum ether and the crystalline product is isolated by filtration with suction.
This gives 20.9 g (99% of theory) of 4-ethylaminocarbonyl-2-methylthiophene-3-sulfon-amide of melting point 185° C.

The compounds of the formula (11) listed in the table below were prepared analogously to example (II-1):

TABLE 2
Examples of compounds of the formula (II)

| | |
|---|---|
| | (II) |
| 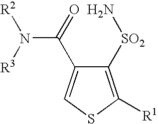 | |

| Ex.-No. | R¹ | R² | R³ | Melting point (° C.) |
|---|---|---|---|---|
| II-2 | CH₃ | H | H | 253 |
| II-3 | CH₃ | CH₃ | CH₃ | 190 |
| II-4 | CH₃ | CH₃ | H | 209 |
| II-5 | CH₃ | n-propyl | H | 188 |
| II-6 | CH₃ | i-propyl | H | 196 |
| II-7 | CH₃ | 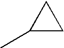 | H | 209 |
| II-8 | CH₃ | —CH₂CH₂CH₂CH₂— | | 134 |
| II-9 | CH₃ | —CH₂CH₂CH₂CH₂CH₂— | | 189 |
| II-10 | CH₃ | 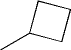 | H | 223 |
| II-11 | CH3 | 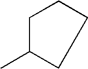 | H | 198 |
| II-12 | CH3 | 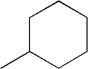 | H | 192 |
| II-13 | CH3 | 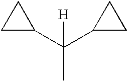 | H | 95 |
| II-14 | CH3 | 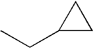 | H | 174 |

### WORKING EXAMPLES

### Example A

### Post-emergence Test

Seeds of mono- and dicotyledonous weed and crop plants are placed in sandy loam soil in wood fiber pots, covered with soil and cultivated in a greenhouse under good growth conditions. 2 to 3 weeks after sowing, the trial plants are treated at the one-leaf stage. The test compounds, formulated as wettable powders (WP), are sprayed at various dosages with a water application rate of 800 I/ha (converted) and with addition of 0.2% of wetting agent onto the green parts of the plants. After the trial plants have stood in the greenhouse for about 3 weeks under optimum growth conditions, the effect of the products is scored visually in comparison to untreated controls (herbicidal effect in percent (%): 100% effect =plants have died, 0% effect=like control plants).

In this test, for example, the compounds of preparation examples 8, 23 and 25 are highly effective against weeds.

TABLE A1
Post-emergence test/greenhouse

| Active | | | | | | | | |
|---|---|---|---|---|---|---|---|---|
| compound of | Application | | | | | | | |
| preparation | rate (g of | | | | | | | |
| example No | ai/ha) | Amaranthus | Lolium | Setaria | Sinapis | Stellaria | Cyperus | Echinochloa |
| (8) | 80 | 80 | — | — | 90 | 70 | — | — |
| (23) | 80 | 90 | 90 | 80 | 90 | 90 | 60 | 90 |
| (25) | 80 | 80 | 90 | 80 | 80 | 90 | 90 | 90 |

1. A compound of the formula (I) 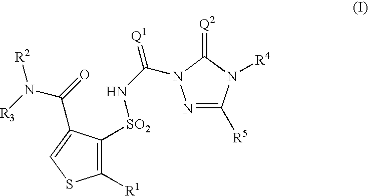
in which
Q¹ is O (oxygen) or S (sulfur),
Q² is O (oxygen) or S (sulfur),
R¹ is hydrogen, cyano, nitro, halogen, is in each case optionally cyano-, halogen- or C₁-C₄-alkoxy-substituted alkyl, alkoxy, alkoxycarbonyl, alkylthio, alkylsulfinyl or alkylsulfonyl having in each case 1 to 6 carbon atoms in the alkyl group, or is in each case optionally cyano- or halogen-substituted alkenyl, alkynyl, alkenyloxy or alkynyloxy having in each case 2 to 6 carbon atoms in the alkenyl or alkynyl group,
R² is hydrogen, is in each case optionally cyano-, halogen-, C₁-C₄-alkylthio-, C₁-C₄-alkylsulfinyl-, C₁-C₄-alkylsulfonyl- or C₁-C₄-alkoxy-substituted alkyl, alkoxycarbonyl, alkylsulfonyl having in each case 1 to 10 carbon atoms in the alkyl group, or is in each case optionally cyano-, halogen- or C₃-C₆-cycloalkyl-substituted cycloalkyl or cycloalkylalkyl having 3 to 6 carbon atoms in the cycloalkyl ring and 1 to 3 carbon atoms in the alkyl group,
R³ is hydrogen or is alkyl having 1 to 6 carbon atoms, or
R² and R³ together are optionally branched alkanediyl having 3 to 7 carbon atoms, where in the alkanediyl chain optionally one methylene group is replaced by an oxygen or nitrogen atom,
R⁴ is hydrogen, hydroxyl, amino, cyano, is C₂-C₁₀-alkylideneamino, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine- and/or bromine-substituted alkenyl or alkynyl having in each case 2 to 6 carbon atoms, is in each case optionally fluorine-, chlorine-, bromine-, cyano-, C₁-C₄-alkoxy- or C₁-C₄-alkoxy-carbonyl-substituted alkoxy, alkylamino or alkylcarbonylamino having in each case 1 to 6 carbon atoms in the alkyl group, is alkenyloxy having 3 to 6 carbon atoms, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkylamino or cycloalkylalkyl having in each case 3 to 6 carbon atoms in the alkyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl- and/or C₁-C₄-alkoxy-substituted aryl or arylalkyl having in each case 6 or 10 carbon atoms in the aryl group and, if appropriate, 1 to 4 carbon atoms in the alkyl moiety, and
R⁵ is hydrogen, hydroxyl, mercapto, amino, cyano, fluorine, chlorine, bromine, iodine, is optionally fluorine-, chlorine-, bromine-, cyano-, C₁-C₄-alkoxy-, C₁-C₄-alkyl-carbonyl- or C₁-C₄-alkoxy-carbonyl-substituted alkyl having 1 to 6 carbon atoms, is in each case optionally fluorine-, chlorine-and/or bromine-substituted alkenyl or alkynyl having in each case 2 to 6 carbon atoms, is in each case optionally fluorine-, chlorine-, cyano-, C₁-C₄-alkoxy- or C₁-C₄-alkoxy-carbonyl-substituted alkoxy, alkylthio, alkylamino or alkylcarbonylamino having in each case 1 to 6 carbon atoms in the alkyl group, is alkenyloxy, alkynyloxy, alkenylthio, alkynylthio, alkenylamino or alkynylamino having in each case 3 to 6 carbon atoms in the alkenyl or alkynyl group, is dialkylamino having in each case 1 to 4 carbon atoms in the alkyl groups, is in each case optionally fluorine-, chlorine-, bromine-, cyano- and/or C₁-C₄-alkyl-substituted cycloalkyl, cycloalkenyl, cycloalkyloxy, cycloalkylthio, cycloalkylamino, cycloalkylalkyl, cycloalkylalkoxy, cycloalkylalkylthio or cycloalkylalkylamino having in each case 3 to 6 carbon atoms in the cycloalkyl or cycloalkenyl group and, if appropriate, 1 to 4 carbon atoms in the alkyl moiety, or is in each case optionally fluorine-, chlorine-, bromine-, cyano-, nitro-, C₁-C₄-alkyl-, trifluoromethyl-, C₁-C₄-alkoxy- and/or C₁-C₄-alkoxy-carbonyl-substituted aryl, arylalkyl, aryloxy, arylalkoxy, arylthio, arylalkylthio, arylamino or arylalkylamino having in each case 6 or 10 carbon atoms in the aryl group and, if appropriate, 1 to 4 carbon atoms in the alkyl moiety
or a salt of a compound of the formula (I).

2. The compound as claimed in claim 1, wherein
R¹ is hydrogen, cyano, fluorine, chlorine, bromine, is in each case optionally cyano-, fluorine-, chlorine-, methoxy- or ethoxy-substituted methyl, ethyl, n- or i-propyl, n-, i-, s- or t-butyl, methoxy, ethoxy, n- or i-propoxy, methoxycarbonyl, ethoxycarbonyl, n- or i-propoxycarbonyl, methylthio, ethylthio, n- or i-propylthio, methylsulfinyl, ethylsulfinyl, methylsulfonyl or ethylsulfonyl, or is in each case optionally cyano-, fluorine- or chlorine-substituted propenyl, butenyl, propynyl, butynyl, propenyloxy, butenyloxy, propynyloxy or butynyloxy,
R² is hydrogen, is in each case optionally cyano-, halogen-, methoxy- or ethoxy-substituted methyl, ethyl, n- or i-propyl, n-, i-, s- or t-butyl, methoxycarbonyl, ethoxycarbonyl, n-propoxycarbonyl, i-propoxycarbonyl, methylsulfonyl or ethylsulfonyl, or is in each case optionally cyano- or halogen-substituted cyclopropyl, cyclobutyl, cyclopentyl, cyclohexyl, cyclopropylmethyl, cyclobutylmethyl, cyclopentylmethyl, cyclohexylmethyl, dicyclopropylmethyl, dicyclobutylmethyl, dicyclopentylmethyl or dicyclohexylmethyl or -C(C₁-C₄-alkyl)₂-CH₂-S(O)ₙ-(C₁-C₄-alkyl) where n=0, 1 or 2,
R³ is hydrogen, methyl, ethyl, n- or i-propyl, or
R² and R³ together are trimethylene (propane-1,3-diyl), tetramethylene (butane-1,4-diyl), pentamethylene (pentane-1,5-diyl) or -CH₂-CH₂-O-CH₂-CH₂-,
R⁴ is hydrogen, hydroxyl, amino, is in each case optionally fluorine-, chlorine-, cyano-, methoxy- or ethoxy-substituted methyl, ethyl, n- or i-propyl, n-, i-, s- or t-butyl, is in each case optionally fluorine-, chlorine-and/or bromine-substituted ethenyl, propenyl, butenyl, propynyl or butynyl, is in each case optionally fluorine-, chlorine-, cyano-, methoxy- or ethoxy-substituted methoxy, ethoxy, n- or i-propoxy, n-, i-, s- or t-butoxy, methylamino, ethylamino, n- or i-propylamino, n-, i-, s- or t-butylamino, is propenyloxy or butenyloxy, is dimethylamino or diethylamino, is in each case optionally fluorine-, chlorine-, methyl- and/or ethyl-substituted cyclopropyl, cyclobutyl, cyclopentyl, cyclohexyl, cyclopropylamino, cyclobutylamino, cyclopentylamino, cyclohexylamino, cyclopropylmethyl, cyclobutylmethyl, cyclopentylmethyl or cyclohexylmethyl, dicyclopropylmethyl, dicyclobutylmethyl, dicyclopentylmethyl or dicyclohexylmethyl, or is in each case optionally fluorine-, chlorine-, methyl-, trifluoromethyl- and/or methoxy-substituted phenyl or benzyl, and
R⁵ is hydrogen, hydroxyl, mercapto, amino, cyano, fluorine, chlorine, bromine, is in each case optionally fluorine-, chlorine-, cyano-, methoxy-, ethoxy-, n- or i-propoxy-, acetyl-, propionyl-, n- or i-butyroyl-, methoxycarbonyl-, ethoxycarbonyl-, n- or i-propoxycarbonyl-substituted methyl, ethyl, n- or i-propyl, n-, i-, s- or t-butyl, is in each case optionally fluorine-, chlorine- and/or bromine-substituted ethenyl, propenyl, butenyl, ethynyl, propynyl or butynyl, is in each case optionally fluorine-, chlorine-, cyano-, methoxy-, ethoxy-, n- or i-propoxy-, methoxycarbonyl-, ethoxycarbonyl-, n- or i-propoxycarbonyl-substituted methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, acetylamino or propionylamino, is propenyloxy, butenyloxy, ethynyloxy, propynyloxy, butynyloxy, propenylthio, butenylthio, propynylthio, butynylthio, propenylamino, butenylamino, propynylamino or butynylamino, is dimethylamino, diethylamino or dipropylamino, is in each case optionally fluorine-, chlorine-, methyl- and/or ethyl-substituted cyclopropyl, cyclobutyl, cyclopentyl, cyclohexyl, cyclopentenyl, cyclohexenyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethylthio, cyclo-butylmethylthio, cyclopentylmethylthio, cyclohexylmethylthio, cyclo-propylmethylamino, cyclobutylmethylamino, cyclopentylmethylamino or cyclohexylmethylamino, or is in each case optionally fluorine-, chlorine-, bromine-, methyl-, trifluoromethyl-, methoxy- or methoxycarbonyl-substituted phenyl, benzyl, phenoxy, benzyloxy, phenylthio, benzylthio, phenylamino or benzylamino, or a sodium, potassium, magnesium, calcium, ammonium, C₁-C₄-alkyl-ammonium, di-(C₁-C₄-alkyl)ammonium, tri-(C₁-C₄-alkyl)ammonium, tetra-(C₁-C₄-alkyl)ammonium, tri-(C₁-C₄-alkyl)sulfonium, C₅- or C₆-cycloalkyl-ammonium or di-(C₁-C₂-alkyl)benzylammonium salt of this compound.

3. The compound as claimed in claim 1, wherein
Q¹ is O (oxygen),
Q² is O (oxygen),
R¹ is methyl, ethyl, n- or i-propyl,
R² is hydrogen, methyl, ethyl, n- or i-propyl or cyclopropyl,
R³ is hydrogen or methyl, or
R² and R³ together are tetramethylene (butane-1,4-diyl) or pentamethylene (pentane-1,5-diyl),
R⁴ is methyl, ethyl, n- or i-propyl or cyclopropyl, and
R⁵ is methoxy, ethoxy, n- or i-propoxy, n-, i-, s- or t-butoxy.

4. A process for preparing compounds as claimed in claim 1, which comprises reacting substituted thiophene-3-sulfonamides of the formula (II) 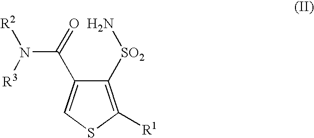
in which
R¹, R² and R³ are as defined in claim 1,
with substituted triazolin(ethi)ones of the formula (III) 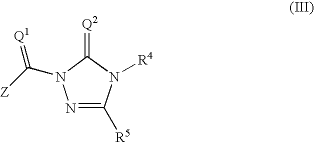
in which
Q¹, Q², R⁴ and R⁵ are as defined in claim 1 and
Z is halogen, alkoxy, aryloxy or arylalkoxy,
if appropriate in the presence of a reaction auxiliary and if appropriate in the presence of a diluent,
and, if appropriate, converting the compounds of the formula (I) obtained by the process by customary methods into salts.

5. A method for controlling unwanted vegetation, which comprises allowing at least one compound as claimed in any of claims 1 to 3 to act on unwanted plants, parts of plants and/or their habitat.

6. The use of at least one compound as claimed in any of claims 1 to 3 for controlling unwanted plants.

7. A herbicidal composition, which comprises one or more compounds as claimed in any of claims 1 to 3 and customary extenders and/or surfactants.
